(19) United States
(12) Patent Application Publication
(10) Pub. No.: US 2015/0299760 A1
(43) Pub. Date: Oct. 22, 2015

(54) SYSTEMS AND METHODS FOR MONITORING BIOLOGICAL FLUIDS

(71) Applicant: OSLO UNIVERSITETSSYKEHUS HF, Oslo (NO)

(72) Inventors: Knut Erik Hovda, Oslo (NO); Petter Urdal, Nesbru (NO); Gaut Gadeholt, Oslo (NO); Dag Jacobsen, Oslo (NO)

(21) Appl. No.: 14/443,817

(22) PCT Filed: Nov. 20, 2013
(86) PCT No.: PCT/IB2013/003203

§ 371 Date: May 19, 2015

Related U.S. Application Data
(60) Provisional application No. 61/728,931, filed on Nov. 21, 2012.

(51) Int. Cl. C12Q 1/32; A61K 31/415; A61K 31/045

(52) U.S. Cl. CPC C12Q 1/32; A61K 31/045; A61K 31/415; G01N 2333/90203; G01N 2333/904; G01N 2800/709

(57) ABSTRACT

The present disclosure relates to compositions and methods for diagnosis, research, and screening for chemicals in biological fluids (e.g., related to methanol poisoning, ethanol levels, and ethylene glycol poisoning). In particular, the present disclosure relates to point of care systems and methods for detecting formic acid or formate, ethanol, ethylene glycol, and other clinically relevant chemicals in biological fluids.

### FIELD OF THE INVENTION

The present disclosure relates to compositions and methods for diagnosis, research, and screening for chemicals in biological fluids (e.g., related to methanol poisoning, ethanol levels, and ethylene glycol poisoning). In particular, the present disclosure relates to point of care systems and methods for detecting formic acid or formate, ethanol, ethylene glycol, and other clinically relevant chemicals in biological fluids.

### BACKGROUND OF THE INVENTION

Methanol poisoning affects thousands each year, of which a large proportion (15-50%) die and many are left permanently blind or have brain damage. In a recent eruption in the Czech Republic, more than 40 dead and more than 120 methanol poisoned are this far reported.

Early diagnosis is essential for successful treatment in methanol poisoning. Diagnosis is difficult, and many never get a definitive diagnosis because methanol analyzes are seldom available. In addition, samples must often be transmitted over great distances for analytical response, after which the patient may already have died or left the hospital without a diagnosis. Patients admitted with a metabolic acidosis without increased S-formate have metabolic acidosis from other causes than methanol poisoning and require different treatment.

What is needed are inexpensive, accurate methods for early diagnosis, which allows treatment to be administered when it is effective. In particular need are systems that can be utilized bedside for point of care analysis.

### SUMMARY OF THE INVENTION

The present disclosure relates to compositions and methods for diagnosis, research, and screening for chemicals in biological fluids (e.g., related to methanol poisoning, ethanol levels, and ethylene glycol poisoning). In particular, the present disclosure relates to point of care systems and methods for detecting formic acid or formate, ethanol, ethylene glycol, and other clinically relevant chemicals in biological fluids.

For example in some embodiments, the present invention provides an assay device (e.g., for detection or the presence, absence, or level, of a toxin or metabolite thereof), comprising: a test strip comprising a) a dehydrogenase enzyme (e.g., formate dehydrogenase, alcohol dehydrogenase, or glycerol dehydrogenase; b) an indicator dye; and c) NAD+. In some embodiments, the test strip further comprises semicarbazide in combination with the dehydrogenase enzyme. The present invention is not limited to a particular indicator dye. In some exemplary embodiments, the indicator dye is (3-(4,5-dimethylthiazol-2-yl)-2,5-diphenyltetrazolium bromide (MTT). The present invention is not limited to a particular material for construction of the test strip. Examples include, but are not limited to, nitrocellulose membranes, nylon membranes, or mixed polymer membrane CQ (IPOC). In some embodiments, the test strip further comprises a sample application pad. In some embodiments, the test strip further comprises a carbohydrate (e.g., trehalose and/or dextran). In some embodiments, the test strip further comprises a surfactant (e.g., BioTerge AS 40). In some embodiments, the test strip further comprises an oxidizing agent (e.g. oxone). In some embodiments, the test strip further comprises bovine serum albumin and/or diaphorase. In some embodiments, the test trip in encased in a housing (e.g., plastic housing) comprising at least one viewing window.

Additional embodiments provide a kit, comprising any of the aforementioned assay devices. In some embodiments, the kit comprises a first test strip comprising formate dehydrogenase and NAD+; and a second test strip comprising alcohol dehydrogenase, and NAD+. In some embodiments, the test strip further comprises semicarbazide in combination with the dehydrogenase enzyme.

Further embodiments provide the use of any of the aforementioned kits to detect a toxin or a metabolite thereof (e.g., formic acid, ethanol, or ethylene glycol) in a biological sample.

Embodiments of the present invention provide a system, comprising: any of the aforementioned kits; and an apparatus or device for detection of NADH (e.g., blood glucose meter or flow through assay).

In further embodiments, the present invention provides a method for detecting a toxin or a metabolite thereof in a biological sample from a subject, comprising: a) contacting a biological sample with a dehydrogenase enzyme that dehydrogenates the toxin or metabolite thereof and NAD+ such that the toxin or metabolite thereof reacts with the dehydrogenase and NAD to generate NADH; and b) detecting NADH. The present invention is not limited to a particular toxin or metabolite. Examples include, but are not limited to, methanol, formic acid, ethanol, or ethylene glycol. The present invention is not limited to a particular dehydrogenase enzyme. Examples include, but are not limited to, formate dehydrogenase, alcohol dehydrogenase, or glycerol dehydrogenase. In some embodiments, the method further comprises contacting the biological sample with semicarbazide. In some embodiments, the biological sample is blood (e.g., whole blood), serum, plasma, or urine. In some embodiments, the dehydrogenase enzyme and the NAD+ are embedded in a test strip (e.g., constructed of a synthetic material). In some embodiments, NADH is detected spectrophotometrically, using a blood glucose meter, using diaphorase and MTT, or using a flow through assay.

In some embodiments, the presence of formic acid in the biological sample is indicative of methanol poisoning in the subject. In some embodiments, the method further comprises the step of treating the subject for methanol poisoning when formic acid is present in the biological sample. In some embodiments, the treatment is ethanol or fomepizole. In some embodiments, ethanol is administered at a concentration of 70-130 mg/dl. In some embodiments, the method further comprises the step of monitoring the subject for levels of ethanol in the biological sample during treatment. In some embodiments, the method is completed in three hours or less (e.g., two hours or less, one hour or less, 30 minutes or less, 15 minutes or less, or 5 minutes or less).

In some embodiments, the present invention provides a method for detecting formic acid in a biological sample from a subject, comprising: a) contacting a biological sample with formate dehydrogenase and NAD+ such that formic acid in the biological sample reacts with said formate dehydrogenase and NAD to generate NADH; and b) detecting the NADH.

The present invention further provides a method for detecting ethanol in a biological sample from a subject, comprising: a) contacting a biological sample with alcohol dehydrogenase and optionally semicarbazide and NAD+ such that ethanol in the biological sample reacts with alcohol dehydrogenase and optionally semicarbazide and NAD to generate NADH; and b) detecting the NADH.

The present invention additionally provides a method for detecting ethylene glycol in a biological sample from a subject, comprising: a) contacting a biological sample with glycerol dehydrogenase and NAD+ such that ethylene glycol in the biological sample reacts with glycerol dehydrogenase and NAD to generate NADH; and b) detecting the NADH.

Additional embodiments of the present disclosure are provided in the description and examples below.

### DESCRIPTION OF THE FIGURES

FIG. 1 shows a schematic of detection of formic acid/formate.

FIG. 2 shows a flow chart of exemplary evaluation of a patient admitted with a suspect methanol poisoning or a patient admitted with a metabolic acidosis of unknown origin.

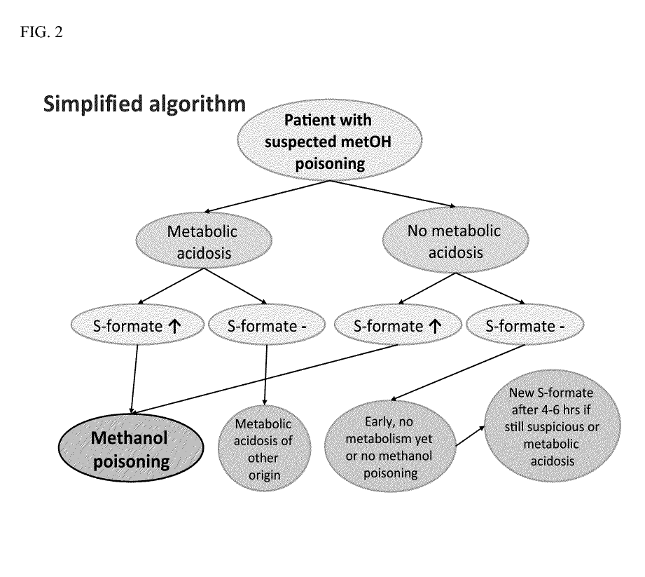

FIG. 3 shows a flow chart of exemplary evaluation of a patient admitted with a suspect methanol poisoning or a patient admitted with a metabolic acidosis of unknown origin.

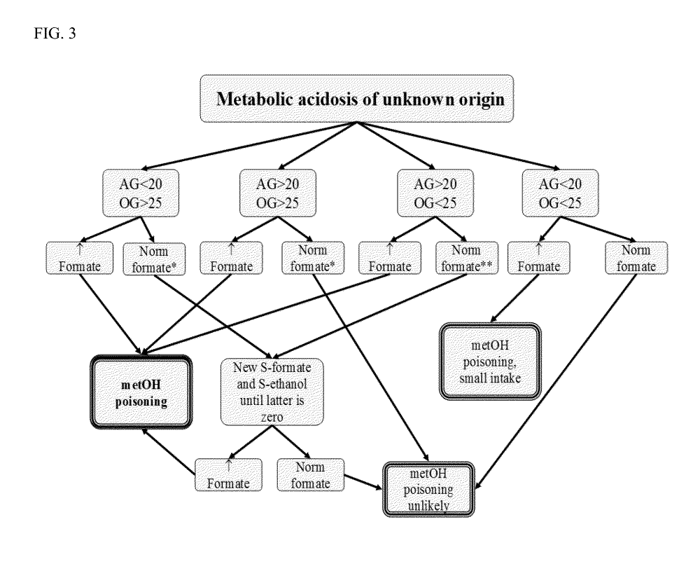

FIG. 4 shows color development at different formate concentrations.

FIG. 5 shows formate level versus color algorithm.

FIG. 6 shows formate measured versus formate added.

FIG. 7 shows a calibration curve for formate in serum.

FIG. 8 shows formate measured in human serum with portable test strip reader (3 repeats).

FIG. 9 shows a calibration curve for formate in whole blood.

FIG. 10 shows formate measured in whole blood with portable test strip reader (3 repeats).

FIG. 11 shows a calibration curve for ethanol in buffer solution.

FIG. 12 shows ethanol measured vs ethanol added.

FIG. 13 shows a calibration curve for ethanol in whole blood.

FIG. 14 shows measurement of ethanol in whole blood with lab-made test strip reader.

FIG. 15 shows reading of formate test strips on channel 1 of a portable colorimeter.

FIG. 16 shows reading of formate test strips on channel 3 of a portable colorimeter.

### DEFINITIONS

Unless defined otherwise, all terms of art, notations and other scientific terms or terminology used herein have the same meaning as is commonly understood by one of ordinary skill in the art to which this disclosure belongs. Many of the techniques and procedures described or referenced herein are well understood and commonly employed using conventional methodology by those skilled in the art. As appropriate, procedures involving the use of commercially available kits and reagents are generally carried out in accordance with manufacturer defined protocols and/or parameters unless otherwise noted. All patents, applications, published applications and other publications referred to herein are incorporated by reference in their entirety. If a definition set forth in this section is contrary to or otherwise inconsistent with a definition set forth in the patents, applications, published applications, and other publications that are herein incorporated by reference, the definition set forth in this section prevails over the definition that is incorporated herein by reference.

As used herein, “a” or “an” means “at least one” or “one or more.”

As used herein, the terms “detect”, “detecting” or “detection” may describe either the general act of discovering or discerning or the specific observation of a detectable composition.

The term “dry reagent test strip” refers to an analytical device in the form of a test strip, in which a test sample fluid, suspected of containing an analyte, is applied to the strip (which is frequently made of bibulous materials such as paper, nitrocellulose, and cellulose). The test fluid and any suspended analyte can flow along the strip to a reaction zone in which the analyte (if present) interacts with a detection agent to indicate a presence, absence and/or quantity of the analyte.

The term “sample application area” refers to an area where a fluid sample is introduced to a test strip, such as a dry reagent test strip described herein or other assay device. In one example, the sample may be introduced to the sample application area by external application, as with a dropper or other applicator. In another example, the sample application area may be directly immersed in the sample, such as when a test strip is dipped into a container holding a sample. In yet another example, the sample may be poured or expressed onto the sample application area.

The term “solid support” or “substrate” means material which is insoluble, or can be made insoluble by a subsequent reaction. Numerous and varied solid supports are known to those in the art and include, without limitation, nitrocellulose, the walls of wells of a reaction tray, multi-well plates, test tubes, polystyrene beads, magnetic beads, membranes, microparticles (such as latex particles), and sheep (or other animal) red blood cells. Any suitable porous material with sufficient porosity to allow access by reagents and a suitable surface affinity to immobilize reagents and/or analyte is contemplated by this term. For example, the porous structure of nitrocellulose has excellent absorption and adsorption qualities for a wide variety of reagents. Nylon possesses similar characteristics and is also suitable. Microporous structures are useful, as are materials with gel structure in the hydrated state.

Further examples of useful solid supports include: natural polymeric carbohydrates and their synthetically modified, cross-linked or substituted derivatives, such as agar, agarose, cross-linked alginic acid, substituted and cross-linked guar gums, cellulose esters, especially with nitric acid and carboxylic acids, mixed cellulose esters, and cellulose ethers; natural polymers containing nitrogen, such as proteins and derivatives, including cross-linked or modified gelatins; natural hydrocarbon polymers, such as latex and rubber; synthetic polymers which may be prepared with suitably porous structures, such as vinyl polymers, including polyethylene, polypropylene, polystyrene, polyvinylchloride, polyvinylacetate and its partially hydrolyzed derivatives, polyacrylamides, polymethacrylates, copolymers and terpolymers of the above polycondensates, such as polyesters, polyamides, and other polymers, such as polyurethanes or polyepoxides; porous inorganic materials such as sulfates or carbonates of alkaline earth metals and magnesium, including barium sulfate, calcium sulfate, calcium carbonate, silicates of alkali and alkaline earth metals, aluminum and magnesium; and aluminum or silicon oxides or hydrates, such as clays, alumina, talc, kaolin, zeolite, silica gel, or glass (these materials may be used as filters with the above polymeric materials); and mixtures or copolymers of the above classes, such as graft copolymers obtained by initializing polymerization of synthetic polymers on a pre-existing natural polymer.

As used herein, the term “sample” is used in its broadest sense. In one sense, it is meant to include a specimen or culture obtained from any source, as well as biological and environmental samples. Biological samples may be obtained from animals (including humans) and encompass fluids, solids, tissues, and gases. Biological samples include blood (e.g., whole blood), blood products, such as plasma, serum, urine, saliva, sputum, and the like. Such examples are not however to be construed as limiting the sample types applicable to the present invention.

### DETAILED DESCRIPTION OF THE INVENTION

The present disclosure relates to compositions and methods for diagnosis, research, and screening for chemicals (e.g., toxins or metabolites thereof) in biological fluids (e.g., related to methanol poisoning, ethanol levels, and ethylene glycol poisoning). In particular, the present disclosure relates to point of care systems and methods for detecting formic acid or formate, ethanol, ethylene glycol, and other clinically relevant chemicals in biological fluids.

Formic acid is the toxic (poisonous) metabolite of methanol, and without the formation of this methanol would not be toxic to humans (d'Alessandro et al., Env. Health Perspectives 102:168 1994; Hovda et al., J. Analytical Toxicology 29 2005; each of which is herein incorporated by reference in its entirety). Treatment of methanol poisoning utilizes inhibitors of the metabolism of methanol to formic acid.

Very few options for detecting methanol poisoning are available. Methanol analyzes are expensive and not easily accessible (only a few centers in Norway are performing them, in New York, analysis takes several days and in the developing world, it often takes several weeks if it at all is possible). Alternative indirect methods exist (Osmolality measurements), but they are nonspecific, and almost never available outside the Western world.

Embodiments of the present disclosure provide solutions for the lack of rapid (e.g., less than several hours, or less than several minutes), cost effective testing for methanol poisoning. In some embodiments, the present invention provides simplified methods for detecting clinically relevant chemicals in biological fluids (e.g., formic acid, formate, ethanol, or ethylene glycol) that utilize a modified version of commercially available blood glucose monitoring systems.

In some embodiments, the present invention provides systems and methods for detection of formic acid to detect methanol poisoning, ethanol levels, or ethylene glycol levels. The systems and methods described herein are simple, inexpensive, rapid, and utilize existing hardware.

### I. Assay Devices, Kits, and Systems

Embodiments of the present disclosure provide assay devices comprising test strips for flow or capillary assays (e.g., alone or in kit or systems). In some embodiments, a test strip or other dry chemistry system where the biological fluid flows onto the dry reagents is utilized (See e.g., U.S. Pat. Nos. 4,774,192 and 4,877,580; each of which is herein incorporated by reference in its entirety).

For example, in some embodiments, test strips are generated using the methods described in the experimental section. The order of absorption of the constituents of the dry chemistry reagent system into the substrate utilized for the test strip is generally dictated by considerations involving chemical compatibly and/or other factors relating to solubility in a common solvent.

In some embodiments, the test strip of the present invention comprises a porous substrate such as a membrane. The porous substrate is preferably impregnated with dry chemical reagents, preferably in a defined reaction zone, that allow detection of an analyte of interest. In some embodiments, the porous substrate in encased in a housing comprising at least one viewing window. In some embodiments, the porous substrate slides within the housing so that it can be viewed through the viewing window and a portion of the substrate extends beyond the housing so that is may be grasped by the user and slid within the housing and/or removed from the housing. In operation of the device, a fluid sample (such as a bodily fluid sample) is placed in contact with the porous substrate. In some embodiments, the device also includes a sample application area (or reservoir) to receive and temporarily retain a fluid sample of a desired volume. In some embodiments, the sample application area facilitates application of a sample to the porous substrate, preferably at sample receptive surface of the porous substrate and adjacent to the reaction zone containing the dry chemistry reagents. The fluid components of the sample pass through the substrate matrix when applied to the porous substrate. In this process, an analyte in the sample (e.g., formate, ethanol, ethylene glycol) can specifically interact with the reagents (e.g., dry chemical reagents deposited using the methods described herein), participate in a chemical reaction, and generate a detectable signal. Optional wash steps can be added at any time in the process, for instance, following application of the sample.

In preferred embodiments, the sample receptive surface is essentially impermeable to cells and particulate matter, but allows diffusion of the analyte into the porous substrate so that the analyte may come into contact with the dry chemistry reagents. In some embodiments, the sample is applied to the sample receptive surface of the porous substrate, allowing for adsorption of the fluid fraction of the sample into the matrix of the porous substrate and detection of an indicator molecule. In some embodiments, the indicator molecule provides for colorimetric quantitation (e.g., semi-quantitative measurement) of the amount of the analyte of interest (e.g., formate) in the sample. In some embodiments, the interaction of the analyte of interest with the reagents in the reaction zone produces a characteristic set of color values that correlate with the presence of specific assay values for a particular analyte. In some embodiments, the assay devices further comprise a color comparator including a plurality of different color fields arranged in an ordered, preferably linear, succession, the color of each field connoting a particular assay value for the analyte. In some embodiments, the color comparator is arranged on the housing so that the porous membrane may be moved in relation to the color comparator to match the color of the reaction zone to the corresponding color on the color comparator to connote a particular assay value for the analyte. In some embodiments, the color comparator is provided separately (e.g., on a separate strip) and the particular assay value for the analyte is obtained by comparing the color comparator to the reaction zone on the porous substrate. In some embodiments, where the porous membrane comprises a sample receptive surface, the device may be preferably inverted so that the color is read from the side opposite of the sample receptive surface. In some embodiments, the porous substrate or the porous substrate within the housing can also be inserted into a reflectance meter, a photometer or a fluorometer; and, the reporter molecule measured and compared with a standard curve for the analyte of interest. The instrument will then report a value based upon its observation and comparison with a standard.

In some embodiments, the porous substrate is conditioned by treatment with a first solution containing protein, glucose, dextrin or dextrans, starch, polyethylene glycol (PEG), polyvinyl pyrolidone (PVP), or an equivalent. The purpose of such conditioning is two-fold: (a) to effectively reduce the void space within the matrix of the substrate and, (b) to assist or promote the absorption of the fluid fraction of the biological sample. In some embodiments, the conditioning agent is combined with one or more of the interactive materials of the reagent system and concurrently absorbed into the substrate. Where the conditioning agent is combined with the interactive materials of the reagent composition, its absorption by the substrate will necessarily be preceded by absorption of the indicator molecule.

Where such conditioning of the porous substrate is effected independent of the interactive materials of the reagent system, the substrate is dried under controlled conditions, and then contacted with one or more solutions containing assay components, for example, enzymes, substrates, and indicator (or the chemical precursor of the indicator molecule) dissolved in a suitable buffer.

In some embodiments, the solution also contains a “flow control agent”. This agent modulates the rate of spreading/distribution of the fluid fraction of this sample throughout the matrix of the substrate. It is, thus, effective in the prevention of the chromatographic separation of the reagents within the membrane matrix upon the addition of the fluid sample. Following addition of this third solution, the substrate is air dried for removal of excess fluid, lyophilized and shielded from light.

Once the reagent delivery system has been prepared, the resultant substrate impregnated with dry chemistry reagents is utilized in any one of several test strip configurations specific for the analysis of whole blood or other samples.

Experiments conducted during the course of development of embodiments of the present disclosure screened a variety of color indicators, buffers for dissolving assay reagents, surfactants, and additional agents to improve stability of assay components. While not limiting the present disclosure to particular components, in some embodiments for detection of formate, the color indicator MTT (3-(4,5-dimethylthiazol-2-yl)-2,5-diphenyltetrazolium bromide) is used. In some embodiments, HEPES buffer (pH 8), trehalose and dextran, BioTerge surfactant, and Oxone are utilized to optimize performance.

In some embodiments for detection of ethanol, EPPS buffer pH 8.4 is used and serum albumin (BSA) is used to protect alcohol dehydrogenase enzyme from degrading.

The specific assay reagents deposited on the substrate depend on the analyte to be detected. In some embodiments, the present disclosure finds use in the detection of methanol (e.g., via formate), ethanol, or ethylene glycol. In some embodiments, the assay reagents include a dehydrogenase enzyme (e.g., formate dehydrogenase, alcohol dehydrogenase and optionally semicarbazide, or glycerol dehydrogenase), NAD+, optionally diaphorase, and an indicator dye (e.g., MTT). The particular materials used in a particular assay strip device will depend on a number of variables, including, for example, the analyte to be detected, the sample volume, the desired flow rate and others. In some embodiments, the sample pad receives the sample, and may serve to remove particulates from the sample. In some embodiments, the sample pad is cellulose. Sample pads may be treated with one or more release agents, such as buffers, salts, proteins, detergents, and surfactants. Such release agents may be useful, for example, to promote resolubilization of conjugate-pad constituents, and to block non-specific binding sites in other components of a lateral flow device, such as a nitrocellulose membrane. Representative release agents include, for example, trehalose or glucose (1%-5%), PVP or PVA (0.5%-2%), Tween 20 or Triton X-100 (0.1%-1%), casein (1%-2%), SDS (0.02%-5%), and PEG (0.02%-5%).

The test strips of embodiments of the present disclosure are not limited to use of a particular substrate. The substrates's physical characteristics (tensile strength, thickness, etc.) are of course to be consistent with test strip manufacture; that is, it should have sufficient dimensional stability and integrity to permit sequential absorption and drying of the conditioning agent the reagent cocktail and/or indicator without loss of its physical strength. The physical attributes of the substrate should also preferably provide sufficient durability and flexibility to adapt in automated processes for continuous manufacturing of test strips. The physical characteristics of the substrate should, in addition, be otherwise consistent with the absorption and retention of aqueous fluids in the contemplated environment of use.

The substrate is preferably relatively chemically inert; that is, essentially unreactive toward both the constituents of the chemistry reagent system and toward the constituents of a sample which is to be reacted with the reagent system within the substrate. It is, however, to be anticipated that certain of the inherent qualities of the substrate surface and/or its matrix may exhibit some affinity for a constituent of the reagent system and/or a constituent of the fluid sample. This natural attraction can, in certain instances, be used to advantage to immobilize a constituent of the reagent cocktail and/or sample on or within a portion of the substrate and thereby effect a type of separation or anisotropic distribution of the constituents of the cocktail/sample. This type of separation, based upon natural binding affinity of the substrate, can be used to advantage in clinical chemistry assays.

The substrate's optical properties should also enable effective observation/monitoring of the reaction manifesting indicator species. This requirement would, thus, contemplate that the substrate provide a background of sufficient contrast to permit observation of the indicator species at relatively low concentrations. Where the indicator is a fluorophore, the background fluorescence of the membrane should be minimal or be essentially non-fluorescent at the monitored wavelength of interest.

Where the inherent characteristics of the substrate are not conducive to effective monitoring of an indicator, it may be desirable to introduce a pigment into the dry chemistry reagent system. For example, certain of the membranes which may be potentially suitable for use in this invention can be colored or transparent. The introduction of pigment into the chemistry reagent system provides a suitable background against which to measure the indicator species.

In some preferred embodiments, the substrate utilized the test strips of the present invention is nitrocellulose, nylon, or mixed polymer membrane CQ (IPOC). Further examples of useful substrates include: natural polymeric carbohydrates and their synthetically modified, cross-linked or substituted derivatives, such as agar, agarose, cross-linked alginic acid, substituted and cross-linked guar gums, cellulose esters, especially with nitric acid and carboxylic acids, mixed cellulose esters, and cellulose ethers; natural polymers containing nitrogen, such as proteins and derivatives, including cross-linked or modified gelatins; natural hydrocarbon polymers, such as latex and rubber; synthetic polymers which may be prepared with suitably porous structures, such as vinyl polymers, including polyethylene, polypropylene, polystyrene, polyvinylchloride, polyvinylacetate and its partially hydrolyzed derivatives, polyacrylamides, polymethacrylates, copolymers and terpolymers of the above polycondensates, such as polyesters, polyamides, and other polymers, such as polyurethanes or polyepoxides; and mixtures or copolymers of the above classes, such as graft copolymers obtained by initializing polymerization of synthetic polymers on a pre-existing natural polymer.

It is contemplated that porous substrates described hereinabove are preferably in the form of sheets or strips. The thickness of such sheets or strips may vary within wide limits, for example, from about 0.01 to 0.5 mm, from about 0.02 to 0.45 mm, from about 0.05 to 0.3 mm, from about 0.075 to 0.25 mm, from about 0.1 to 0.2 mm, or from about 0.11 to 0.15 mm.

The surface of a solid support may be activated by chemical processes that cause covalent linkage of an agent (e.g., an assay reagent) to the support. However, any other suitable method may be used for immobilizing an agent to a solid support including, without limitation, ionic interactions, hydrophobic interactions, covalent interactions and the like. The particular forces that result in immobilization of an agent on a solid phase are not important for the methods and devices described herein.

Except as otherwise physically constrained, a substrate may be used in any suitable shapes, such as films, sheets, strips, or plates, or it may be coated onto or bonded or laminated to appropriate inert carriers, such as paper, glass, plastic films, or fabrics.

In some embodiments, assay strip devices of the present invention include a strip of absorbent or porous material (such as a microporous membrane), which, in some instances, can be made of different substances each joined to the other in zones, which may be abutted and/or overlapped. In some examples, the absorbent strip can be fixed on a supporting non-interactive material (such as nonwoven polyester), for example, to provide increased rigidity to the strip.

In some embodiments, a fluid sample (or a sample suspended in a fluid) is introduced to the strip at the sample receptive surface, for instance by dipping or spotting. A sample is collected or obtained using methods well known to those skilled in the art. The sample containing the analyte to be detected may be obtained from any biological source. Examples of biological sources include blood serum, blood plasma, urine, spinal fluid, saliva, fermentation fluid, lymph fluid, tissue culture fluid and ascites fluid of a human or animal. The sample may be diluted, purified, concentrated, filtered, dissolved, suspended or otherwise manipulated prior to the assay to optimize the results. The fluid migrates distally through all the functional regions of the strip. The final distribution of the fluid in the individual functional regions depends on the adsorptive capacity and the dimensions of the materials used.

Other useful assay device formats which may be adapted for use in the present invention are described in, e.g., U.S. Pat. No. 4,770,853; PCT Publication No. WO 88/08534 and European Patent No. EP-A 0 299 428, and U.S. Pat. Nos. 5,229,073; 5,591,645; 4,168,146; 4,366,241; 4,855,240; 4,861,711; 4,703,017; 5,451,504; 5,451,507; 5,798,273; 6,001,658; and 5,120,643; European Patent No. 0296724; WO 97/06439; and WO 98/36278, all of which are incorporated herein by reference.

In some embodiments, the present invention provides a kit comprising components useful, necessary, or sufficient for measuring toxins or metabolites there of (e.g., formic acid/formate, ethanol, or ethylene glycol) in a biological sample (e.g., blood, plasma, serum, or urine). In some embodiments, kits comprise, consist essentially of, or consist of, a dehydrogenase enzyme (e.g., formate dehydrogenase, alcohol dehydrogenase (optionally in combination with semicarbazide), or glycerol dehydrogenase), indicator dye (e.g., MTT), NAD+, positive control, and directions for use. In some embodiments, the dehydrogenase, NAD+, indicator dye, and any additional components are embedded on a test strip. In some embodiments, kits comprise reagents for identifying multiple analytes (e.g., ethanol and methanol) in a biological sample (e.g., multiple test strips, each of which is specific for a different analyte or a single strip that detects multiple analytes).

In some embodiments, kits are generally portable and provide a simple, rapid, and/or cost-effective way to determine the presence or absence of analytes without the need for laboratory facilities, such as in a point-of-care facility.

In some embodiments, the kits of the present invention include one or more assay devices and optionally a reader or other detection device, as disclosed herein and a carrier means, such as a box, a bag, a satchel, plastic carton (such as molded plastic or other clear packaging), wrapper (such as, a sealed or sealable plastic, paper, or metallic wrapper), or other container. In some examples, kit components will be enclosed in a single packaging unit, such as a box or other container, which packaging unit may have compartments into which one or more components of the kit can be placed. In other examples, a kit includes one or more containers, for instance vials, tubes, and the like that can retain, for example, one or more biological samples to be tested, positive and/or negative control samples or solutions, diluents (such as, phosphate buffers, or saline buffers), detector reagents, and/or wash solutions (such as, buffers, saline buffer, or distilled water).

Other kit embodiments include syringes, finger-prick devices, alcohol swabs, gauze squares, cotton balls, bandages, latex gloves, incubation trays with variable numbers of troughs, adhesive plate sealers, data reporting sheets, which may be useful for handling, collecting and/or processing a biological sample. Kits may also optionally contain implements useful for introducing samples into a sample chamber of an assay device, including, for example, droppers, Dispo-pipettes, capillary tubes, rubber bulbs (e.g., for capillary tubes), and the like. Still other kit embodiments may include disposal means for discarding a used assay device and/or other items used with the device (such as patient samples, etc.). Such disposal means can include, without limitation, containers that are capable of containing leakage from discarded materials, such as plastic, metal or other impermeable bags, boxes or containers.

In some embodiments, a kit of the present invention will include instructions for the use of an assay device. The instructions may provide direction on how to apply sample to the test device, the amount of time necessary or advisable to wait for results to develop, and details on how to read and interpret the results of the test. Such instructions may also include standards, such as standard tables, graphs, or pictures for comparison of the results of a test. These standards may optionally include the information necessary to quantify analyte using the test device, such as a standard curve relating intensity of signal or number of signal lines to an amount of analyte therefore present in the sample.

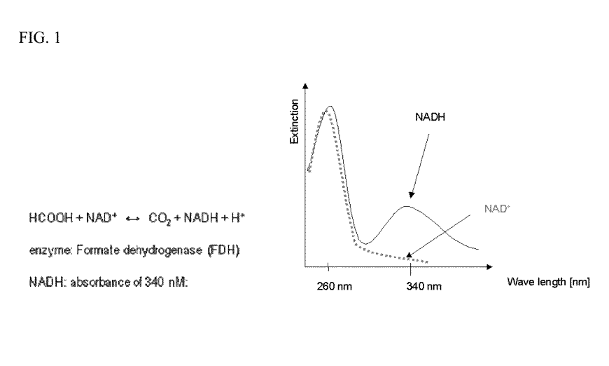

In some embodiments, the present disclosure provides systems comprising the assay devices described herein; and a detection device. In some embodiments, currently available blood glucose meters are utilized to detect levels of toxins or metabolites thereof (e.g., formic acid levels or the presence or absence of formic acid or formate, ethanol levels, or ethylene glycol levels (e.g., using the chemistry described herein)). For example, in some embodiments, commercially available blood glucose meters (e.g., from Life Scan, Milpitas, Calif.; Abbott Laboratories, Abbott Park, Ill.; Roche Diagnostics, Indianapolis, Ind.)). For example, in some embodiments, glucose meters that use the chemistry described in FIG. 1 of the coupling of glucose dehydrogenase to the conversion of NAD to NADH (See e.g., U.S. Pat. No. 6,312,888; herein incorporated by reference in its entirety) is utilized. In the presence of NADH, with the enzyme diaphorase as a catalyst, the yellow MTT ((3-(4,5-Dimethylthiazol-2-yl)-2,5-diphenyltetrazolium bromide, a yellow tetrazole) is transformed to the purple formazan which is quantified photometrically. If the calibration is in millimoles per liter, all reactions that produce one molecule of NADH per molecule use the same calibration as the glucose measurement.

Such meters utilize a test strip (e.g., those described herein). Blood is applied to the test strip. The test strip is inserted into the meter, which then measures the production of NADH (e.g., spectrophotometrically). In such embodiments, the glucose dehydrogenase is replaced with formate dehydrogenase, alcohol dehydrogenase (optionally in combination with semicarbazide), or glycerol dehydrogenase. The chemistry described above is then utilized to measure formic acid/formate in blood or urine.

The present invention is not limited to the use of blood glucose meters for detection. In some embodiments, the chemistry described herein is applied in capillary microfluidic platforms (See e.g., Chem. Soc. Rev., 2010, 39, 1153-1182; herein incorporated by reference in its entirety), paper-based devices (See e.g., Anal. Chem. 2009, 81, 8447-8452; herein incorporated by reference in its entirety), laboratory test strip readers, or filter paper.

### II. Methods

In some embodiments, the devices, kits, systems and methods described herein find use in monitoring methanol outbreaks, ethylene glycol poisoning, and ethanol levels in the field. In some embodiments, systems, kits, and methods find use in the developing world where the ability to rapidly and inexpensively detect methanol poisoning in the field is particularly useful. The systems and methods described herein are able to provide a definitive diagnosis in 15-120 seconds using a drop of blood without relying on laboratory equipment.

The symptoms of ethanol intoxication, metabolic acidosis, and methanol poisoning can be difficult to distinguish between. In addition, some incidents of methanol poisoning are the result of ethanol that is contaminated with methanol. It is important to be able to rapidly distinguish between acidosis, ethanol intoxication and methanol poisoning in order to administer appropriate treatment. Accordingly, in some embodiments, the systems and methods described herein find use in distinguishing between exposure to methanol and ethanol or metabolic acidosis of unknown or other origin in a subject. Test strips for detection of methanol (e.g., test strips for detection of formic acid/formate) and test strips for detection of ethanol are used to rapidly provide a diagnosis.

In some embodiments, the systems and methods described herein are used to monitor treatment for methanol poisoning. In some embodiments, methanol poisoning is treated by administration of ethanol or fomepizole. During treatment with ethanol, it is important to closely monitor blood levels of ethanol to keep them in an appropriate therapeutic range (e.g., 70-130 mg/dl). The test strips of embodiments of the present disclosure find use in the rapid detection of ethanol levels in blood and are suitable for use in monitory treatment with ethanol. In some embodiments, formate levels are concurrently measured with ethanol levels to confirm that treatment is effective.

### EXPERIMENTAL

The following examples are provided in order to demonstrate and further illustrate certain preferred embodiments and aspects of the present disclosure and are not to be construed as limiting the scope thereof.

### Example 1

### Reagents

1. NAD (nicotinamide dinucleotide) commercially available from, for example, Sigma and many other companies).
2. Formate dehydrogenase from Roche (catalog no 244 678, contains 80 U or other concentration)
3. A phosphate buffer pH=7.5 such as 0.1M phosphate buffered saline (PBS) pH=7.5

### Preparation of Reagents for Use:

Both NAD and formate dehydrogenase are sold in dry state and are stable for extended periods.
1. Dissolve approx 0.2 g of NAD in 30 ml buffer. (Reagent 1)
2. Dissolve the content of one bottle formate dehydrogenase (80 U) in 5 ml buffer (Reagent 2)
Both reagents are stable at 4-8° C. for approx 4-5 days and frozen reagents (preferentially at −40° C. or below) are stable for many months.

### Analysis

1. Mix 1 part sample (serum or blood), 10 parts reagent 1 and 5 parts reagent 2.
2. Incubate for 5-10 minutes at 37° C. or at 20° C. for 7-10 min.
3. Measure absorbance in a photometer at 340 nm.

### Evaluation of Result

1. The reagent itself (in the absence of sample) gives an absorbance of approx 0.1-0.3.
2. Formate is normally present in serum and gives an extra absorbance of less than 0.1.
3. An absorbance more than 0.1 above that of the reagent is pathological and indicates increased concentration of formate. A serum containing 5 mmol/1 formate shows typically an absorbance of 0.7-0.8, that is 0.6-0.7 absorbance units above the absorbance of the reagent.

In some embodiments, a serum containing known amounts of formate is analyzed together with the patient serum samples as a control and to calibrate formate concentration in the patient samples analyzed.

### Example 2

This example describes the development of a colorimetric dry-reagent test strip for measuring formate in the range of 0-20 mM in buffer solutions with detection limit ≦2 mM by simple dip and read procedure. The strip also finds use in measuring formate in for example, whole blood and serum.

The developed formate test strip is a dry-reagent, self-dosing, “dip-and-read”, colorimetric test device, which contains in dry state all reagents needed for measuring formate. The following reagents were used: Formate Dehydrogenase (Roche Applied Sciences), Diaphorase (Sigma D2197), MTT (Sigma M2128), NAD (Sigma N1636). Other components were also included in the formate formula for better enzymes stability and strip performance.

The strip was made as follows:

a. Single reagent mixture was prepared by making HEPES buffer pH 7.9 first.

b. Enzymes were added from 100 U/mL stock solutions (in the same buffer).

c. All other ingredients were added to the mixture as dry powders and stirred gently.

d. PVP/PES mixed polymer porous membrane was impregnated with the reagent mixture by simple dipping process.

e. Membrane was dried for 1 hour at 45° C.

f. Membrane was attached to the white polystyrene plastic support through the double side adhesive.

The procedure for semi-quantitative measurement of formate is simple and fast. The procedure involves dipping the strip into a test solution and comparing the color of the strip to a color chart. The analysis takes about 2 minutes. Blood was applied on one side of the strip and results were registered from the other.

The developed test strip can also be used for quantitatively measuring formate with an appropriate reflectometer.

Four different color indicators, all diaphrase substrates, were evaluated, including MTT, INT, NBT and 2,6-dichlorophenolindophenol. None of them worked better than MTT (3-(4,5-dimethylthiazol-2-yl)-2,5-diphenyltetrazolium bromide. MTT was used in this study.

Several biological and others pH buffers were evaluated including phosphate, borate, imidazole, tricine, Tris, bis-Tris, bis-Tris propane, Epps and HEPES in the pH range 7.5-8.5. HEPES (pH 8) was found more preferable as it provided lower background color and better stability. Concentrations of buffer, indicator and enzymes activity were optimized for the best test strip performance and formate detection in 1-20 mM range.

Screening of additional components for better enzyme stability and strip performance was done. These components were polymers such as polyvinyl alcohol, polyethylene glycol, polyvinylpyrrolidone, dextran, BSA, Metocell, Klucel and Gantez; and carbohydrates such as: sucrose, lactitol, lactose, cyclodextrin, sorbitol and trehalose. Most of the carbohydrates improved test strips stability under the 40° C. drying conditions. A combination of trehalose and dextran additives was selected.

Screening of different surfactant to improve test strip wetting properties was performed. Brige 35, BioTerge AS 40, Triton X-100, Tween 20, benzalconium, sodium cholate, Igepal 210, Chemal LA-9, Tetronic 1307, Pluronic L64, Standapol ES-1, Rhodasuron 870. Most of the detergents had a negative effect on background color. BioTerge AS 40 didn't affect background color and was selected as an additive to the test strip formula.

Oxidizing agent additives such as Chloramine T, sodium nitite and oxone were evaluated as NADH scavengers to improve test strip color distinction in 1-20 mM formate range. Oxone was selected to improve colors distinction and its concentration was optimized.

Several porous membranes were screened as a matrix for the formate test strip and the best was used in this study. It includes: nitrocellulose Hi-Flow membrane (Millipore), Nitrobind and BioTrace (Pall), nylon membranes Immunodyne ABC, Biodyne A and Biodyne B (Pall), PES membranes for blood separation Vivid GR, GX, GF (Pall), Cytosep 1660 (Pall), mixed polymer membranes X, NX, CQ and SG from (IPOC). Nitrocellulose and nylon membranes demonstrated quite good performance and low background color development. However, mixed polymer membrane CQ (IPOC) was selected as it finds use for blood separation and can be used for a whole blood testing device.

A color chart for semi-quantitative formate measurement in formate standard solutions was developed. Formate standards were prepared using physiological buffer solution. Quantitative measurement of formate was done with Evik lab-made reflectometer. Samples of the formate test strip were prepared and evaluated as described below.

Semi-quantitative analysis with color chart and quantitative analysis with color analyzer (Evik lab) were conducted for evaluation performance of formate test strip.

—Semi-Quantitative Formate Measurement

The procedure for semi-quantitative measuring of formate is simple and fast. The procedure involves dipping a strip into test solution for 5 seconds, taking it out and comparing color of the strip to color chart in 2 minutes. The strip changes color from yellow to purple when formate level in solution is changed from 0 to 20 mM or above. The color chart for measuring formate has 4 color blocks indicating 0, 1, 5, and 20 mM. If the color of the strip matches one of the color blocks, formate level can be read under the corresponding color block. Intermediate number for formate level should be used if color of the strip is between neighboring color blocks. See Table 1 for the results obtained.

TABLE 1
Results of semi-quantitative measuring of formate with color chart.

| | Formate in Solution | | | | |
|---|---|---|---|---|---|
| | 1 mM | 2 mM | 3 mM | 4 mM | 5 mM |
| Strip color | Match | Slightly | Between 1 | Slightly | Match |
| | 1 mM | higher than | and 5 mM | lower than | 5 mM |
| | color | 1 mM | color | 5 mM | color |
| | block | color | blocks | color | block |
| | | block | | block | |
| Formate | 1 mM | ~2 mM | ~3 mM | ~4 mM | 5 mM |
| measured | | | | | |

—Instrumental Readings of Formate Test Strips

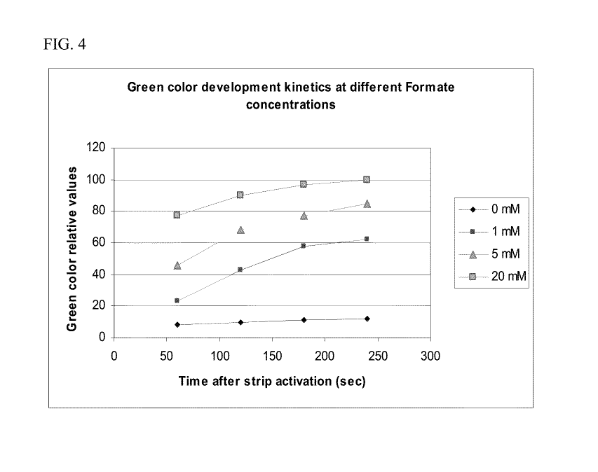

This study was done to demonstrate feasibility of quantitative measuring of formate and for preliminary determination of accuracy and limit of detection. RGB (Red, Green, Blue) numbers were collected for several formate standards with Evik color analyzer in 1, 2 and 3 minutes after strip activation. Results are shown in FIG. 4.

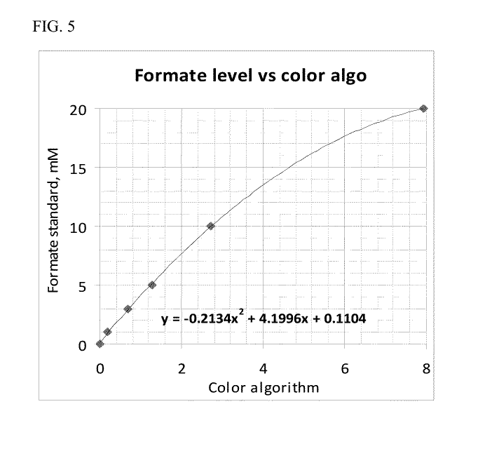

Color algorithm (special RGB combination) and equation for calculation of formate level were determined Results are shown in FIG. 5.

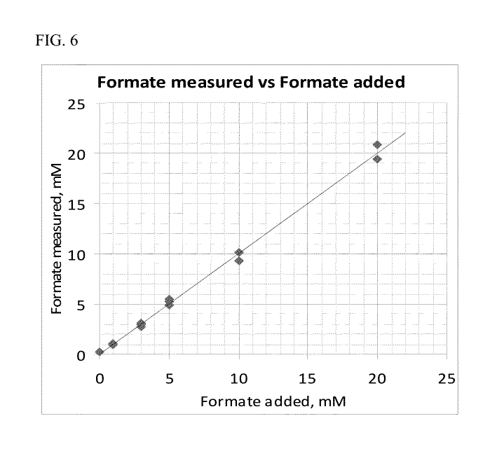

Formate solutions (0, 1, 3, 5, 10 and 20 mM in PBS buffer) were prepared. 2-3 strips (repeats) were activated in each of these solutions and formate concentration was determined with Evik color analyzer in 2 minutes after strip activation. Good correlation was observed between sample formate and measured formate levels. Results are shown in FIG. 6. A similar result was observed in 1 minute after strip activation but with higher data scattering at that time.

The results show that the developed test strip prototype can be used for quantitative measuring of formate in the 0-20 mM range with limit of detection less than 1 mM.

### Example 3

This example describes the detection of formate in serum and whole blood.

### Formate in Serum

### Materials Used:

For this study the early developed Formate test strip formula and pooled human serum from Cedarlane laboratories were used.

### Preparation of Serum Samples:

Serum samples having 0, 1, 3, 5, 10 and 20 mM formate were prepared by addition of formate stock solutions (0.1 M and 0.5 M, pH 7.5) to the human serum.

### Strip Activation:

20 μL of serum sample were put on the top surface of the formate reagent pad. After 5 seconds excess of liquid was removed from the strip surface. Color of the strip was measured in 2 minutes after strip activation.

### Visual Evaluation:

Visual readings revealed clear color distinction between all the formate levels in serum −0, 1, 3, 5, 10 and 20 mM. Color of the strip changed from light yellow-greenish to green to blue in the 0-20 ppm formate range. The greenish color hue was due to the yellow serum color.

### Instrumental Strip Readings:

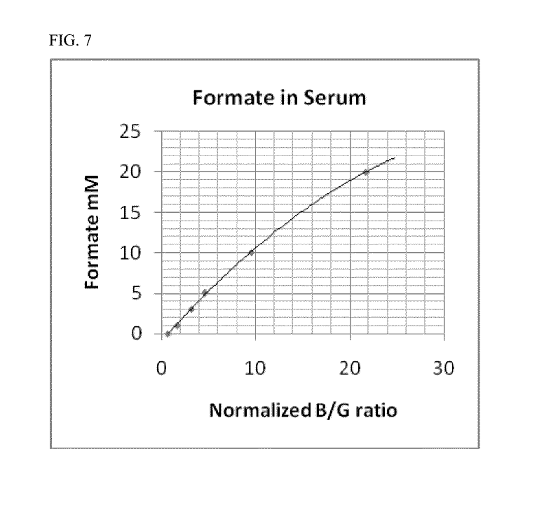

Instrumental reading was done with laboratory test strip reader made by Evik Diagnostics, Inc (Canada). The colorimeter was calibrated with standard formate solutions in serum. RGB numbers were collected for 0, 1, 3, 5, 10 and 20 mM formate. Color algorithm was developed on the base of this data (see FIG. 7). It was observed that serum itself as well as test strips activated in serum had yellow color hue. Thus, to eliminate potential interference from serum, special correction of color algorithms was performed. Normalized RGB numbers obtained for strips activated in “zero formate” serum samples were subtracted from RGB numbers obtained for serum samples having formate. Normalized B/G ratio was chosen as a color algorithm for formate in serum (FIG. 7).

### Results of Quantitative Formate Measuring in Human Serum:

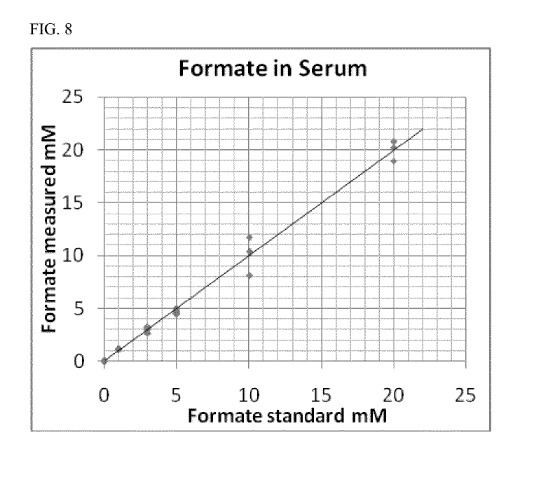

Serum samples having 0, 1, 3, 5, 10 and 20 mM formate were prepared and three repeats were done for each formate level. As shown in FIG. 8, good correlation between formate measured and formate standards was observed.

### Formate in Whole Blood

### Materials Used:

For measuring formate in blood, formate test strips as described herein were used. Samples of sheep whole blood were obtained from Cedarlane laboratories. Two types of blood samples were used: citrate-treated blood and defibrinated blood.

### Preparation of Blood Samples:

Blood samples having 0, 1, 3, 5, 10 and 20 mM formate were prepared by addition of formate stock solutions (0.1 M and 0.5 M, pH 7.5) to the whole blood in plastic container.

### Evaluation of the Early Developed Test Strip for Measuring:

The same strip design as was developed for buffer solution was tested first. 20 μL of the blood sample was put on the top surface of the reagent pad. After 5 seconds excess of blood was removed from the strip surface. Color of the strip was visually observed in 2 minutes after strip activation.

It was observed that test strip activated in “zero ppm” formate samples had rather intense reddish color. Clear increase in blue color hue was seen at 5, 10 and 20 ppm formate. However, no or very small color distinction was observed for the strips activated in 0, 1 and 3 ppm formate.

### Development of New Aperture-Based Formate Test Strip.

Several different designs for whole blood application were evaluated. For example, stacks of two reagent and blood separation membranes were evaluated. Membranes for vertical and lateral blood separation were evaluated. Some combinations looked promising but significant results variability was observed due to the manual device assembly. Non-uniform contact interface between two membranes resulted in non-uniform pad wetup and color.

The most reproducible results were obtained by applying the formate reagent formula directly on porous membrane for vertical blood separation. The formate reagent paper was attached to the perforated pieces of plastic support through the double sided adhesive film. Thus both surfaces of the reagent paper were exposed to the environment.

### Activation of the Aperture-Based Formate Test Strip:

10 μL of the blood sample were put on the top surface of the reagent pad. After 15 seconds excess of blood was removed from the strip surface. Color of the strip was read through the aperture on the opposite (bottom) side of the strip in 2 minutes after strip activation.

### Visual Readings of the Aperture-Based Formate Test Strip:

Visual readings were done by comparison of colors of the strip activated in two neighboring blood sample in the range of 0-20 mM formate. Visual readings revealed clear color distinction between all the formate levels in blood having 0, 1, 3, 5, 10 and 20 mM formate.

### Instrumental Readings of the Aperture-Based Formate Test Strip:

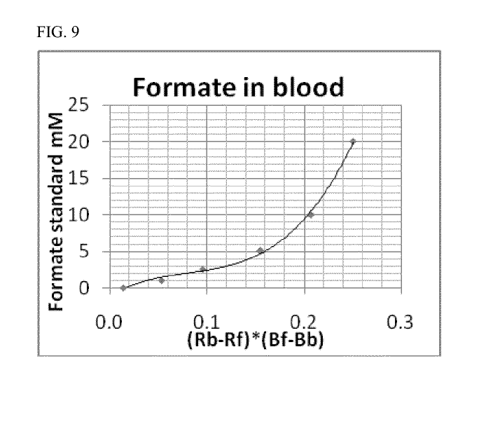

Instrumental readings were done with a portable test strip reader made by Evik Diagnostics, Inc. It was observed that small reddish color hue was still observed when the strips were activated in “zero ppm” blood samples. Thus to eliminate this interference from blood, correction of color algorithms was done. Normalized RGB numbers obtained for blank strips activated in “zero formate” blood samples were subtracted from RGB numbers obtained for blood samples having formate. Normalized (Rb−Rf)*(Bf−Bb) equation was chosen as a color algorithm for formate in blood (FIG. 9).

### Results of Quantitative Formate Measuring in Whole Blood:

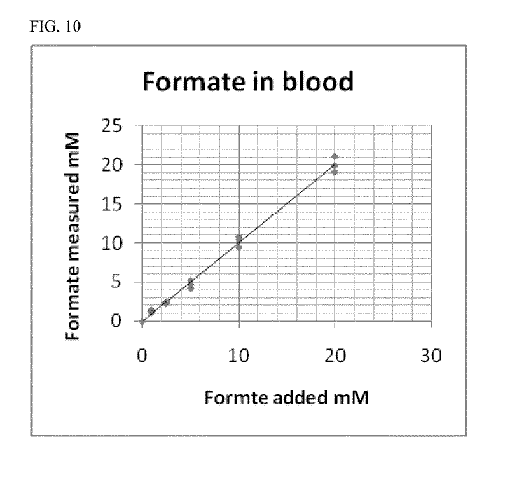

Blood samples having 0, 1, 3, 5, 10 and 20 mM formate were prepared and three repeats were done for each formate level. As shown in FIG. 10, good correlation between formate measured in whole blood and formate standards was observed.

### Example 4

This example describes detection of ethanol in buffer solutions using test strips.

Replacement of formate dehydrogenase (FDH) for alcohol dehydrogenase (ADH) in the early-developed formate test strip formulation.

Ethanol strips were prepared the same way as the early-developed formate test strip, except alcohol dehydrogenase (ADH) replaced formate dehydrogenase (FDH) in the test strip formulation.

The ethanol strip was activated by dipping in ethanol buffer solutions of 0, 0.25, 0.5 and 1.5 mM ethanol. No color development was observed in 1-10 minutes after activation of the strip. ADH didn't work in these conditions.

Development of New Ethanol Test Strip. E-02A Formula.

### Materials Used:

Alcohol Dehydrogenase was obtained from Sigma-Aldrich. The same Diaphorase and MTT as for the formate test strip were used. Supporting components were adjusted to provide visual color changes on Immunodyne nylon porous membrane (Pall).

### Preparation of Ethanol Test Strip:

Test strips were prepared by dipping porous nylon membrane into the impregnation mixture and drying for 30 min at 40° C. Reagent pads were attached to the plastic support through the double sided adhesive film.

### Preparation of Ethanol Samples:

Ethanol samples having 0, 0.125, 0.25, 0.5, 0.75 and 1.5 g/L ethanol in PBS buffer were prepared by addition of ethanol stock solutions (10 g/L) to the PBS buffer.

### Test Strip Activation:

The strips were dipped in ethanol solutions for 5 seconds and removed. Excess of liquid was removed with filter paper. Color of the strip was read in 2 minutes after strip activation with laboratory test strip reader made at Evik Diagnostics, Inc. (Canada).

### Instrumental Readings of the Ethanol Test Strip:

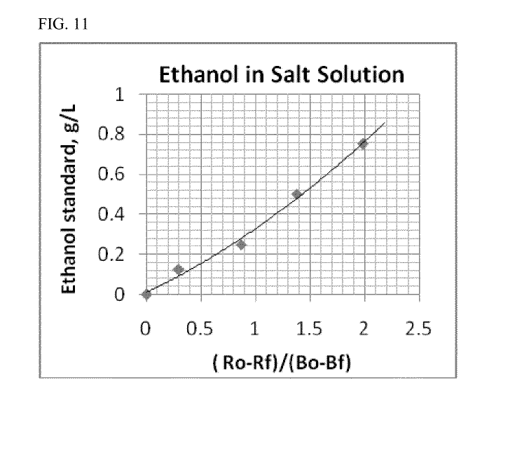

Normalized (Ro−Rf)/(Bo−Bf) ratio was chosen as a color algorithm for ethanol in buffer solution. Color algorithm was increased in the 0-0.75 g/L ethanol. The strip was calibrated in the 0-0.75 g/L ethanol range (See FIG. 11).

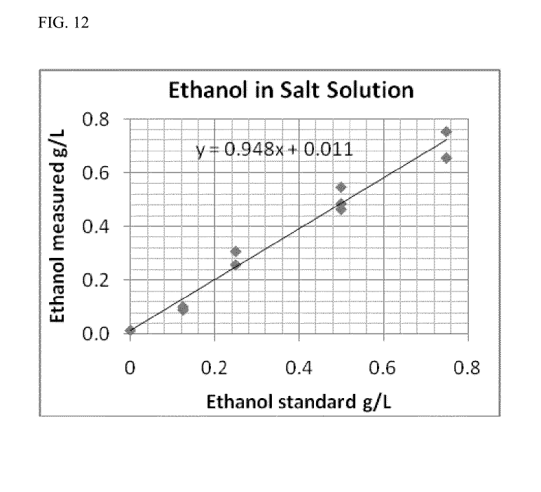

Using this calibration curve ethanol samples of 0, 0.125, 0.25, 0.5 and 0.75 g/L ethanol in PBS buffer were measured with the EDI strip reader. The result is shown in FIG. 12. Good correlation was observed between Ethanol added and ethanol measured. These results indicate that for measuring ethanol in whole blood the blood samples should be diluted at least 1:2 (v/v).

### Example 5

This example describes detection of ethanol in whole blood using test strips.

Evaluation of Ethanol Test Strip (Formula E-2A) with Whole Blood.

It was found that Ethanol test strip (Formula E-2A), which was used for measuring ethanol in buffer solution did not work with whole blood samples. Thus a new formula, compatible with blood separation membrane, was developed and an aperture-based test strip was designed.

### Development of New Aperture-Based Ethanol Test Strip Prototype (Formula E-3B).

### Materials Used:

Alcohol Dehydrogenase was obtained from Sigma-Aldrich. The same Diaphorase and MTT as for the formate test strip were used (Example 2). Supporting components were changed. Samples of sheep whole blood were obtained from Cedarlane laboratories. Two types of blood samples were use: citrate-treated blood and defibrinated blood.

Preparation of New Aperture-Based Ethanol Test Strip. Formula E-3B:

Several different masking agents were evaluated to reduce effect of blood separation membrane material on alcohol dehydrogenase enzyme stability. The pre-treatment of the blood separation membrane with thickening agents (polymers) was evaluated. The addition of bovine serum albumin (BSA) protected alcohol dehydrogenase enzyme from degrading. EPPS buffer pH 8.4 was prepared first and all other reagents were dissolved in this buffer with gentle stirring as shown in Table 2.

TABLE 2

| | Amount per 1 mL |
|---|---|
| Component | of final mix (mg) |
| Diaphorase Sigma (U) | 12 |
| Alcohol Dehydrogenase Sigma(U) | 12 |
| EPPS | 20 |
| NAD | 0.3 |
| MTT | 1.8 |
| Trehalose | 80 |
| BSA | 5 |

This alcohol reagent formula was applied directly to a porous membrane for vertical blood separation through the impregnation process as described in Example 2 above. Reagent paper was dried in the oven for 30 min at 40° C. The ethanol reagent matrix was attached to the perforated pieces of plastic support through the double-sided adhesive film. Thus both surfaces of the reagent paper were exposed to the environment.

### Preparation of Blood Samples:

Blood samples having 0, 0.125, 0.25, 0.5, 0.75 and 1.5 g/L ethanol were prepared by addition of ethanol stock solutions (100 g/L) to the whole blood in plastic container.

### Activation of the Aperture-Based Ethanol Test Strip:

10 μL of the blood sample were put on the top surface of the reagent pad. After 15 seconds excess of blood was removed from the strip surface. The color of the strip was read through the aperture on the opposite (bottom) side of the strip in 3 minutes after strip activation.

### Visual Readings of the Aperture-Based Ethanol Test Strip:

Visual readings were done by comparison of colors of the strip activated in two neighboring blood samples in the range of 0-1.5 g/L ethanol. Visual readings revealed clear color distinction between all the ethanol levels in blood (0, 0.125, 0.25, 0.5, 0.75, 1.0 and 1.5 g/L ethanol).

### Instrumental Readings of the Aperture-Based Ethanol Test Strip:

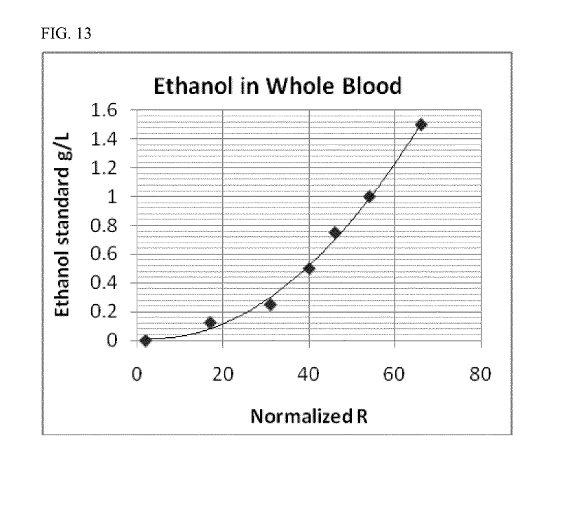

Instrumental reading was done with a laboratory test strip reader made by Evik Diagnostics, Inc. It was observed that a small reddish color hue was observed when the strips were activated in “zero ppm” blood samples. Thus, to eliminate this interference from blood special correction of color algorithms was done. Normalized R numbers obtained for blank strips activated in “zero ethanol” blood samples were subtracted from R numbers obtained for blood samples having ethanol. R value was chosen as a color algorithm for ethanol in blood (FIG. 13).

### Results of Quantitative Measuring of Ethanol in Whole Blood:

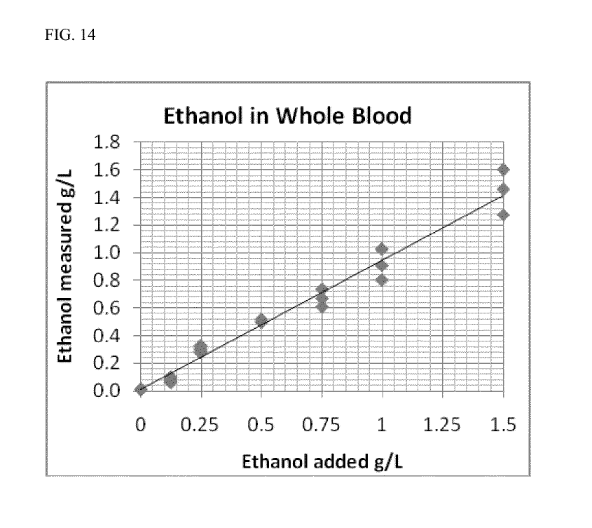

Blood samples having 0, 0.125, 0.25, 0.5, 0.75 and 1.5 g/L ethanol were prepared and three repeats were done for each ethanol level. As shown in FIG. 14, a linear correlation between ethanol measured in whole blood and ethanol added was observed.

### Example 6

This example describes analysis of formate strip assays on a commercially available portable colorimeter.

Water Quality Meter from ITS:

A multichannel portable colorimeter/test strip reader (Pool Check i test strip analyzer) was purchased from ITS Co (USA). This test strip reader was designed for simultaneous measuring six different water-soluble components. The meter is applicable for using regular test strips having specific size of test pad. ITS claimed that their meter is a meter of “medical” quality.

### Approach Used:

The six-channel strip reader was used in order to find out whether one or more channels allow measuring formate with the developed formate test strip. Modified formate test strips having specific size of test pad and test pad position on the strip were prepared. Performance of the prepared strip on each of the channels was evaluated.

### Activation of the Modified Formate Test Strip.

The strip was dipped for 3 seconds in PBS buffer having 0, 1, 3, 10 and 20 mM formate. Excess of liquid was removed by shaking and the strip was put on the strip slot. Color of the strip was read on each channel in 2 minutes after activation of the strip.

### Results Obtained:

It was found that two channels, channel #1 (TC, Total Chlorine) and channel #3 (FC, Free Chlorine) allow measuring formate in the 0-20 mM range. Both ITS strips, TC and FC change color from light yellow to blue to purple in the range of 0-10 ppm TC or FC. This color change is similar to the color range of the formate test strip.

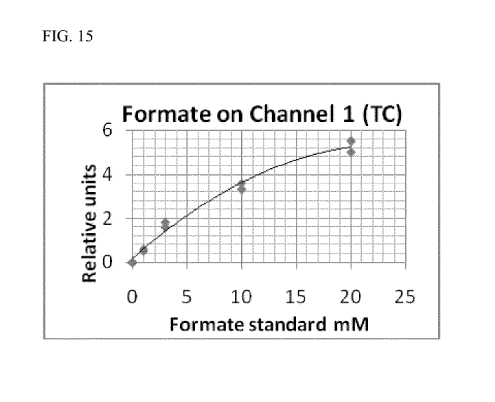

FIG. 15 demonstrates performance of the strip on channel #1 (TC). As shown in FIG. 15 good color distinction (relative units, TC ppm) was observed between all the formate levels (0, 1, 3, 10 and 20 mM formate) when formate strip was evaluated on this channel.

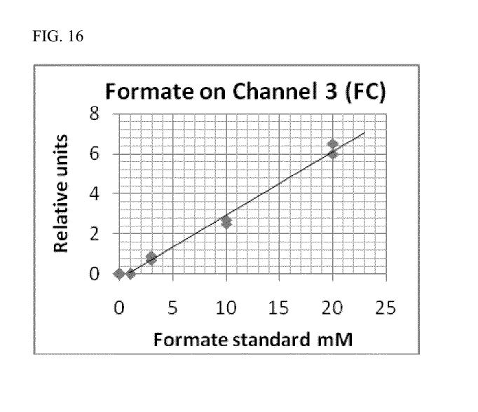

FIG. 16 demonstrates performance of the strip on channel #3 (FC). Color of the strip (relative units, FC ppm) changed almost linear vs. formate standards. In this case, the limit of detection was lower, 1 mM formate was read as 0 mM.

Although a variety of embodiments have been described in connection with the present disclosure, it should be understood that the claimed invention should not be unduly limited to such specific embodiments. Indeed, various modifications and variations of the described compositions and methods of the invention will be apparent to those of ordinary skill in the art and are intended to be within the scope of the following claims.

1.-19. (canceled)

20. A method for detecting methanol, ethanol or ethylene glycol poisoning in a subject, comprising:
a) contacting a biological sample with a dehydrogenase enzyme that dehydrogenates said toxin or metabolite thereof selected from the group consisting of methanol, formic acid, ethanol, and ethylene glycol and NAD+ such that said toxin or metabolite thereof reacts with said dehydrogenase and NAD to generate NADH; and
b) detecting said NADH, wherein the presence of NADH is indicative of methanol, ethanol or ethylene glycol poisoning in said subject.

21. (canceled)

22. The method of claim 20, wherein said dehydrogenase enzyme is selected from the group consisting of formate dehydrogenase when the toxin or metabolite is methanol or formic acid, alcohol dehydrogenase when the toxin is ethanol, and glycerol dehydrogenase when the toxin is ethylene glycol.

23. The method of claim 20, further comprising contacting said biological sample with semicarbazide.

24. The method of claim 20, wherein said biological sample is blood, serum, plasma, or urine.

25. The method of claim 20, wherein said dehydrogenase enzyme and said NAD+ are embedded in a test strip.

26. The method of claim 25, wherein said test strip is selected from the group consisting of nitrocellulose membranes, nylon membranes, and mixed polymer membrane CQ (IPOC).

27. The method of claim 20, wherein said NADH is detected spectrophotometrically.

28. The method of claim 20, wherein said NADH is detected using a blood glucose meter.

29. The method of claim 20, wherein said NADH is detected using diaphorase and MTT.

30. The method of claim 20, wherein said NADH is detected using a flow through assay.

31. The method of claim 20, wherein the presence of formic acid in said biological sample is indicative of methanol poisoning in said subject.

32. The method of claim 31, further comprising the step of treating said subject for methanol poisoning when formic acid is present in said biological sample.

33. The method of claim 32, wherein said treatment is ethanol or fomepizole.

34. The method of claim 33, wherein said ethanol is administered at a concentration of 70-130 mg/dl.

35. The method of claim 32, further comprising the step of monitoring said subject for levels of ethanol is said biological sample during said treatment.

36. The method of claim 20, wherein said method is completed in 1 hour or less.

37. The method of claim 20, wherein said method is completed in 5 minutes or less.

38. A method for detecting formic acid in a biological sample from a subject suspected of having methanol poisoning, comprising:
a) contacting a biological sample with formate dehydrogenase and NAD+ such that formic acid in said biological sample reacts with said formate dehydrogenase and NAD+ to generate NADH; and
b) detecting said NADH, wherein the formation of said NADH is indicative of methanol poisoning in said subject.

39.-41. (canceled)

42. The method of claim 38, further comprising the step of identifying said subject for treatment for methanol poisoning.
